(19) United States
(12) Patent Application Publication
(10) Pub. No.: US 2025/0072812 A1
(43) Pub. Date: Mar. 6, 2025

(54) EEG RECOGNITION METHOD FOR NATURAL HAND MOVEMENT BASED ON TIME-DOMAIN AND FREQUENCY-DOMAIN MULTI-LAYER BRAIN NETWORK

(71) Applicant: SOUTHEAST UNIVERSITY, Jiangsu (CN)

(72) Inventors: Baoguo XU, Jiangsu (CN); Zelin GAO, Jiangsu (CN); Xinhao YANG, Jiangsu (CN); Aiguo SONG, Jiangsu (CN)

(73) Assignee: SOUTHEAST UNIVERSITY, Jiangsu (CN)

(21) Appl. No.: 18/808,099

(22) Filed: Aug. 19, 2024

Related U.S. Application Data
(63) Continuation of application No. PCT/CN2023/127258, filed on Oct. 27, 2023.

(30) Foreign Application Priority Data
Aug. 28, 2023 (CN) 202311088296.2

(51) Int. Cl. A61B 5/374; A61B 5/00

(52) U.S. Cl. CPC A61B 5/374; A61B 5/7239; A61B 5/725; A61B 5/7253; A61B 5/7267

(57) ABSTRACT

Disclosed is an EEG recognition method for a natural hand movement based on a time-domain and frequency-domain multi-layer brain network, including: (1) acquiring a multi-channel EEG signal of the natural hand movement; (2) preprocessing the multi-channel EEG signal, and extracting a δ wave, a θ wave, a α wave, a β wave, and a γ wave at each time point; (3) constructing a time-domain multi-layer brain network using a wSAR model; (4) calculating the frequency-domain multi-layer brain network using the phase-amplitude coupling; (5) combining the time-domain multi-layer brain network and the frequency-domain multi-layer brain network, and performing standardization; and (6) calculating metrics of the time-domain and frequency-domain multi-layer brain network and a super-adjacency matrix of a decomposed time-domain and frequency-domain multi-layer brain network, inputting the same to a two-layer graph convolutional network (GCN), and fusing manual, shallow, and deep features for analysis.

### CROSS-REFERENCE TO RELATED APPLICATION

This application is a continuation of PCT application No. PCT/CN2023/127258, filed on Oct. 27, 2023, which claims a priority benefit to China patent application No. CN202311088296.2 filed on Aug. 28, 2023. The entirety of the above-mentioned patent applications are hereby incorporated by reference herein and made a part of this specification.

### TECHNICAL FIELD

The present disclosure belongs to the technical field of electroencephalogram signal processing, and particularly relates to an EEG recognition method for a natural hand movement based on a time-domain and frequency-domain multi-layer brain network.

### BACKGROUND

A brain-computer interface (BCI) is a method of controlling an external device through an electroencephalogram (EEG) signal, and it acquires and analyzes the electroencephalogram, decodes the electroencephalogram into control commands that can be recognized by a machine, thereby achieving control of the external device. At present, the BCI has been extensively used in the fields of medical rehabilitation and neuroengineering. In the research on the EEG signal of natural hand movements, the fineness of human hand movements and external interference contribute to the complexity of natural actions, and many natural actions can be realized through coordination of many brain regions. However, traditional EEG signal feature extraction methods often consider only individual features of some brain regions, making it difficult for these methods to extract more effective features.

Given the increasing demand for joint analysis of multiple brain regions, brain network analysis has gradually become a main focus in the BCI. Brain network analysis can explore neural connection relationship of a brain from different aspects, such as macro aspect and micro aspect, and can also explore different types of connection modes between brain regions from structural, functional, or causal levels. In recent years, brain network analysis has mainly focused on the analysis of functional connection and causal connectivity.

Although brain network analysis exhibits strong vitality, information represented by a single-layer brain network only reflect a relationships between specific time or specific frequencies, and feature information obtained therefrom is still subjected to significant limitations. Therefore, multi-layer brain network analysis is necessary in current research. Since a definition of the multi-layer brain network analysis is ambiguous, most of the current researches on the multi-layer brain network analysis have ignored the importance of inter-layer connections, resulting in information loss in multi-layer brain networks. Furthermore, many previous researches have only considered the multi-layer brain networks in a frequency domain or dynamic brain networks in a time domain, but ignored the multi-layer brain networks in the time domain, and failed to make full use of the information from the multi-layer brain networks.

### SUMMARY

In order to solve the above problems, the present disclosure discloses an electroencephalogram (EEG) signal recognition method for a natural hand movement based on a time-domain and frequency-domain multi-layer brain network, which introduces a new possibility for inter-layer connections between a time-domain multi-layer brain network and a frequency-domain multi-layer brain network, and also provides a new approach for the comprehensive analysis of the EEG of natural hand movement.

In order to achieve the above objective, the present disclosure adopts a following technical solution:
an EEG recognition method for a natural hand movement based on a time-domain and frequency-domain multi-layer brain network, including following steps:
(1) acquiring a multi-channel EEG signal of the natural hand movement;
(2) preprocessing the multi-channel EEG signal, extracting an EEG signal at each time point by using a sliding time window, and extracting a δ wave, a θ wave, a α wave, a β wave, and a γ wave at the each time point.
(3) processing data of the each time point, and constructing a time-domain multi-layer brain network by taking an electrode as a node, and a directed transfer function (DTF) as an intra-layer connection; Particularly, a method for calculating a time-domain inter-layer connection is as follows: constructing a weighted quadratic autoregressive (wSAR) model: adding a weighted term to an autoregressive (AR) model based on a hypothesis that the closer a time distance is, the greater an impact becomes, and processing a constant term, and calculating parameters of the improved wSAR model as the inter-layer connection.
(4) Processing data of each frequency band, and calculating cross-frequency inter-layer coupling. A method for calculating a frequency-domain inter-layer connection is as follows: performing Hilbert transform on the EEG signal of each frequency, extracting amplitude and phase information, and calculating cross-frequency coupling (CFC) between every two frequencies, and selecting an average value of phase-amplitude coupling (PAC), phase-phase coupling (PPC), and amplitude-amplitude coupling (AAC) as a final inter-layer connection herein.
(5) Combining the time-domain and frequency-domain multi-layer brain networks respectively constructed in the steps (3) and (4) into a 3D standardized form: M=(V, E, ω, l), where V represents a set of points, E represents a set of edges, ω represents a size of an edge, and l represents a position label of a point. In the standardized form, brain regions are abstracted into a z-axis, the time is an x-axis, the frequency is a y-axis, that is, a single-layer network is compressed into one dimension, and as a dimension of the number of layers increases, a number of dimensionalities of the multi-layer brain network increases.
(6) Calculating metrics of the multi-layer brain network in the standardized form obtained in the step (5), including a common multi-layer degree, eigenvector centrality, and temporal flexibility specific to the multi-layer brain network, which are used for a nerve analysis of natural action decoding, and finally, calculating an adjacency matrix, and inputting the standardized form into the GCN for classification and identification.

Further, the step (2) includes:
(2.1) performing low-pass filtering at 100 Hz on the EEG signal, removing outliers, and performing data supplementation by using linear interpolation;
(2.2) removing other bioelectrical components by using an independent component analysis (ICA) (a blind source separation algorithm), and then performing segmentation, baseline correction and common average reference; and
(2.3) performing filtering on the processed EEG signal at 0.3-3 Hz (δ wave), 4-8 Hz (θ wave), 8-13 Hz (α wave), 13-30 Hz (β wave) and 30-45 Hz (γ wave) by using a 4th-order Butterworth IIR filter, and sampling the EEG signal of each frequency by using the sliding time window with a length of 1 s and a step length of 100 ms to obtain a signal value of the corresponding time point.

Further, the step (3) includes:
(3.1) selecting an EEG signal of a certain channel in a certain frequency band, and constructing the wSAR model for the EEG signal:

$${x}_{i + 1}^{j} = {a}_{m}^{j} {x}_{i}^{j} + {a}_{m - 1}^{j} {x}_{i - 1}^{j} + … + {a}_{1}^{j} {x}_{i - m}^{j} + {ka}_{m}^{j} + {ω}^{j} + ε$$

where xᵢ^{j} represents an EEG signal value of a channel j at a moment i, aₘ^{j} represents a model parameter value of the channel j, ω^{j} represents a constant term, which is assumed to be an initial value of influence of each channel, ε represents a noise, a model order is m, and k is a weighted value. When a value of the k is too large, subsequent time terms will lose their meanings, therefore, in order to avoid the situation, k is set to an average value from xᵢ₋ₘ^{j} to xᵢ^{j}, and in order to consider an optimal order, an initial value of the model order m is set to 1 here.
(3.2) Rewriting a formula of the wSAR model as follows:

$$ε = {x}_{i + 1}^{j} - [ {a}_{m}^{j} ( {x}_{i}^{j} + k ) + {a}_{m - 1}^{j} {x}_{i - 1}^{j} + … + {a}_{1}^{j} {x}_{i - m}^{j} + {ka}_{m}^{j} + {ω}^{j} ]$$

a least squares method is adopted, and a partial derivative of ε² to a^{j} is set to equal to 0 to minimize a residual sum of squares.

In order to make the formula expression easier, the wSAR model is denoted in a matrix form as

$${Y}^{j} = [ \begin{matrix}{x}_{i + 1}^{j} \\ {x}_{i + 2}^{j} \\ … \\ {x}_{N}^{j}\end{matrix} ] , {X}^{j} = [ \begin{matrix}{x}_{i}^{j} + k & {x}_{i - 1}^{j} & … & {x}_{i - m}^{j} & 1 \\ {x}_{i + 1}^{j} + k & {x}_{i}^{j} & … & {x}_{i + 1 - m}^{j} & 1 \\ … & … & … & … & 1 \\ {x}_{N - 1}^{j} + k & {x}_{N - 2}^{j} & … & {x}_{N - 1 + m}^{j} & 1\end{matrix} ] , \\ {A}^{j} = [ \begin{matrix}{a}_{m}^{j} \\ … \\ {a}_{2}^{j} \\ {a}_{1}^{j} \\ {ω}^{j}\end{matrix} ] , ε = [ \begin{matrix}{ε}_{1} \\ {ε}_{2} \\ … \\ {ε}_{m}\end{matrix} ]$$

where N is a total number of time points, and a least squares estimation of a parameter matrix A^{j} is expressed as:

$${A}^{j} = {( {X}^{{j}^{T}} {X}^{j} )}^{- 1} {X}^{{j}^{T}} {Y}^{j}$$

(3.3) Obtaining all the parameter matrixes A^{j} by having EEG signals of all the channels in the frequency band calculated in the step (3.2). According to the actual situation, when neither action intention nor action execution is made, all the channels shall have a same preliminary value for an action response, therefore, preliminary values of all the channels need to be aligned. In the method, an average value of the constant term ω^{j} in each of the parameter matrix A^{j} is taken to obtain ω_{avg}, that is

$${ω}_{avg} = \frac{1}{M} {\sum }_{j} {ω}^{j}$$

where M represents a total number of the channels, and ω^{j} is a constant term of a parameter matrix of each of the channels. The step (3.2) is performed again by substituting ω_{avg} back to an original formula of the AR model of all the channels to obtain a final parameter matrix B^{j}.
(3.4) Taking the model order m from 1 to 10, repeating the steps (3.2) and (3.3), and calculating self-covariance functions σ₁, σ₂, . . . , σ₁₀ of samples of each order; and calculating an order capable of making a final prediction error (FPE) reach a minimum according to an FPE criterion, and selecting a parameter matrix B^{j} of the order.
(3.5) Multiplying both sides of an equation of the AR model of all the channels by a time-lag term xᵢ₋ₛ^{j}^{^{T}}, performing z transformation to obtain a system transfer function Hᵢⱼ(ƒ), which represents a transfer relationship from a channel i to the channel j; and then performing normalization to obtain a final directed transfer function (DTF) γᵢⱼ²(ƒ):

$${γ}_{ij}^{2} ( f ) = \frac{{\vert {H}_{ij} ( f ) \vert}^{2}}{{\sum }_{m = 1}^{M} {\vert {H}_{im} ( f ) \vert}^{2}}$$

where Hᵢₘ(ƒ) represents a transfer function from the channel i to a channel m.
(3.6) Performing calculation of the signals of all the frequency bands according to the steps (3.2)-(3.5) to finally construct the time-domain multi-layer brain network: taking electrodes as nodes; taking 60% of a threshold to remove false connections, and regarding the finally obtained γᵢⱼ²(ƒ) as the intra-layer connection, that is, a connection from a node i to a node j in a same layer is taken as γᵢⱼ²(ƒ); 60% of the threshold is taken to remove false connections, and the parameter matrix B^{j} is regarded as the intra-layer connection, that is, a connection between a node i in a k^{th} layer to a node i in an l^{th} layer of a frequency band j is b_{m+1-|k-l|}^{j}.

Further, the step (4) includes:
(4.1) selecting the EEG signal of each frequency, and performing the Hilbert transform on all the time points to obtain an amplitude value and a phase value of the each time point;
(4.2) calculating the PAC, the PPC, and the AAC between every two frequencies:

$$PAC = \vert \frac{1}{n} {\sum }_{i = 1}^{n} {a}_{i}^{H} {e}^{j {∅}_{i}^{L}} \vert$$

where n is a total number of time points in EEG data, aᵢ^{H} represents an amplitude value of a high-frequency band H at the moment i, and Øᵢ^{L} represents the phase of a low frequency band L at the moment i;

$$PPC = \vert \frac{1}{n} {\sum }_{i = 1}^{n} sgn [ Im ( {e}^{j ( {∅}_{i}^{a} - {∅}_{i}^{b} )} ) ] \vert$$

where Øᵢ^{a} and Øᵢ^{b} represent phase values of frequency bands a and b at the moment i, respectively, and an imaginary part and an sgn function are taken to reduce volume conduction effect and the influence of a common source;

$$AAC = Corr ( {A}^{i} , {A}^{j} )$$

where Corr( ) represents a Pearson correlation coefficient, Aⁱ and A^{j} represent the amplitude values of frequency bands i and j, respectively; and
(4.3) calculating the average value of the PAC, the PPC and the AAC as a cross-frequency coupling (CFC) value, taking 60% of the threshold to remove the false connections, and using the CFC value as the inter-layer coupling between corresponding frequencies to form a frequency-domain part of the multi-layer brain network.

Further, the step (5) includes:
the obtained time-domain and frequency-domain multi-layer brain network is written into the standardized form: M=(V, E, ω, l), where the brain regions are the z-axis, the time is the x-axis, and the frequency is the y-axis, V is a point set, including a point label, for example, (“ν₁”, . . . , “ν_{N}”); E represents the edge, for example, (“e₁₂”, . . . , “e_{N(N-1)}”); ω is a connection value of the edge, for example (ω(νᵢ, νⱼ), . . . ); i is a position label of the point ν, which is used to indicate the position of the point, therefore, l is a 2D matrix, for example, ((“ν₁”, 1, 1, 1), . . . , (“ν₂₅”, 2, 5, 6), . . . ), and since the present disclosure has three dimensions, three numbers after the point label constitute coordinates of the point. A reason for standardizing the multi-layer brain network is to make it visualized and facilitate retrieval, and make it convenient for subsequent calculation of metrics and unified processing of a graph convolutional network (GCN).

Further, the step (6) includes:
(6.1) calculating metrics of the time-domain and frequency-domain multi-layer brain network: the multi-layer degree d(i), the eigenvector centrality θ_{jβ}, and a node flexibility F are used for a neurophysiological analysis. Furthermore, manually extracted features are inputted into a last layer of the GCN for weighted fusion of features:

$${d}_{M} ( i ) = {\sum }_{\begin{matrix}j , k ∈ V \\ j , k \neq i\end{matrix}} [ a ( i , j , k ) + a ( j , i , k ) + a ( k , j , i ) ] \\ {ϑ}_{j β} = {λ}^{- 1} {M}_{j β}^{i α} {ϑ}_{j α} \\ {F}_{j} = \frac{1}{N} {\sum }_{i} {f}_{i} \\ Q = {\sum }_{ijkr} [ ( {A}_{ijs} - {γ}_{s} {P}_{ijs} ) δ ( s , k ) + {ω}_{isk} ] δ ( {c}_{ir} , {c}_{is} )$$

where d_{M}(i) is a total node degree of νᵢ, a(i,j,k) is an element in an adjacency tensor of the time-domain and frequency-domain multi-layer brain network; M_{jβ}^{iα} is the adjacency tensor of the time-domain and frequency-domain multi-layer brain network; θ_{jβ} and θ_{jα} are eigenvectors of the tensor corresponding to an eigenvalue λ; ƒᵢ is a number of changes of a node community at the each time point, N is a total number of changes, and Fⱼ is node flexibility of a node νⱼ; Q is a modular function used for community detection, where Aᵢⱼₛ is an adjacency matrix of an s^{th} layer, Pᵢⱼₛ is a relevant zero matrix, γₛ is a resolution parameter, ωᵢₛₖ is a connection value from a node i in the s^{th} layer to a node i in the k^{th} layer, cᵢᵣ and cᵢₛ are set parameters; and δ( ) is a Dirac function;
(6.2) inputting the time-domain and frequency-domain multi-layer brain network into the GCN, where the inputted node signal value is a degree of freedom of each node; and the inputted adjacency matrix is a super-adjacency matrix of a decomposed time-domain and frequency-domain multi-layer brain network. Particularly, a method for processing the super-adjacency matrix of a decomposed time-domain and frequency-domain multi-layer brain network is as follows: since the adjacency matrix of the time-domain and frequency-domain multi-layer brain network is a 3D tensor, and is difficult to be calculated due to a large number of dimensionalities. In order to reduce the computational load, inspired by Cartesian product of graphs, the time-domain and frequency-domain multi-layer brain network is decomposed into sub-networks in two directions, an original 3D adjacency matrix is decomposed into two orthogonal 2D super-adjacency matrices as adjacency matrix inputs to the GCN, and finally feature fusion is performed;
(6.3) a structure of the GCN includes two layers of GCN and one feature classification layer. In order to make full use of information of the time-domain and frequency-domain multi-layer brain network, the two layers of GCN are divided into a shallow layer of GCN and a deep layer of GCN, all graphs output features once after passing through the shallow layer of GCN, output features once again after passing through the deep layer of GCN, and finally, manual, shallow and deep features are fused to obtain final classification results.

The present disclosure has the beneficial effects:
(1) The present disclosure constructs a functional connectivity model of the brain using the time-domain and frequency-domain multi-layer brain network approach. Compared with traditional methods and general brain networks, the approach describes the functional connectivity of the brain during natural hand movements from a more comprehensive perspective, and explains the operational mechanism of the brain from a new perspective.
(2) The present disclosure emphasizes the importance of inter-layer connections of the multi-layer brain network and introduces a new method for calculating the connections using the wSAR model and the mixed cross-frequency coupling, which improves the interpretability of inter-layer connections and enhances the information validity of the multi-layer brain network, providing a new method for improving the decoding of the natural hand movements.
(3) The present disclosure standardizes the time-domain and frequency-domain multi-layer brain network, allowing for a clear visualization the connection modes of the functional brain network, which is conducive to revealing the neural operational mechanism of the brain.
(4) The present disclosure reduces the dimensionality of the adjacency matrix of the time-domain and frequency-domain multi-layer brain network to reduce computational load, and considers manual, shallow, and deep features of the multi-layer brain network while improving the GCN, thereby improving the accuracy of the decoding of natural hand movements while utilizing the information of the multi-layer brain network information more comprehensively.

### BRIEF DESCRIPTION OF THE DRAWINGS

FIG. 1 is a main flow chart of an EEG recognition method for a natural hand movement based on a time-domain and frequency-domain multi-layer brain network according to the present disclosure.

FIG. 2 is a flow chart of constructing a time-domain multi-layer brain network of an EEG recognition method for a natural hand movement based on a time-domain and frequency-domain multi-layer brain network according to the present disclosure.

FIG. 3 is a diagram for standardizing a time-domain and frequency-domain multi-layer brain network of an EEG recognition method for a natural hand movement based on a time-domain and frequency-domain multi-layer brain network according to the present disclosure.

FIG. 4 is a GCN structural diagram of an EEG recognition method for a natural hand movement based on a time-domain and frequency-domain multi-layer brain network according to the present disclosure.

### DETAILED DESCRIPTIONS OF THE EMBODIMENTS

The present disclosure will be further illustrated below with reference to the accompanying drawings and specific embodiments. It should be understood that the following specific embodiments are only used to illustrate the present disclosure, but are not intended to limit the scope of the present disclosure.

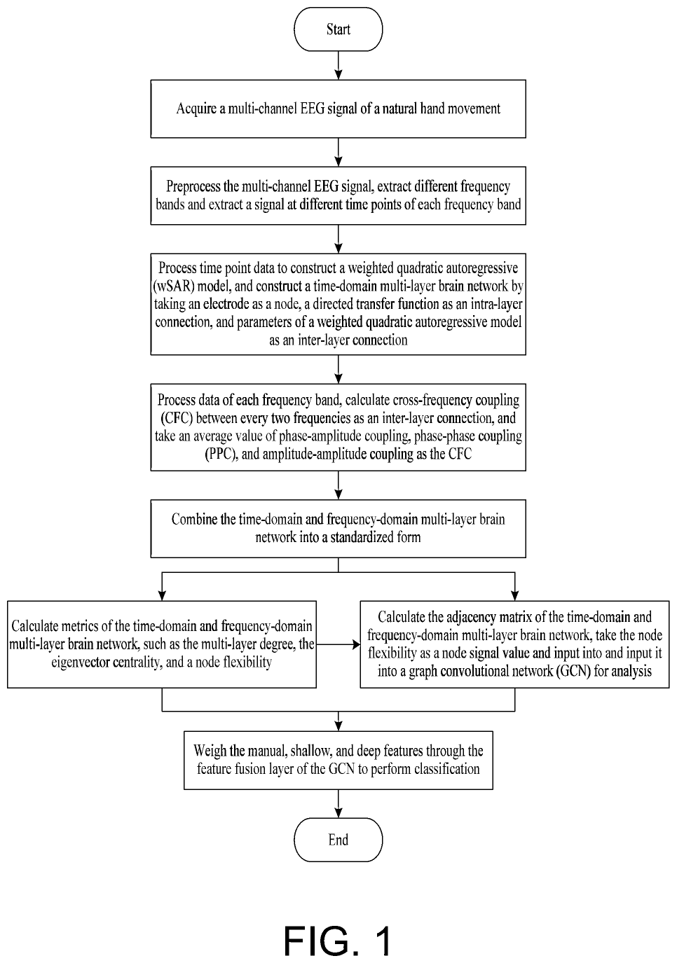

As shown in FIG. 1, the present disclosure provides an electroencephalogram (EEG) signal recognition method for a natural hand movement based on a time-domain and frequency-domain multi-layer brain network, specifically including following steps:
(1) acquiring a multi-channel EEG signal of the natural hand movement;
(2) preprocessing the multi-channel EEG signal, extracting an EEG signal at each time point by using a sliding time window, and extracting a δ wave, a θ wave, a α wave, a β wave, and a γ wave at the each time point.

Specifically, the step (2) includes following sub-steps:
(2.1) performing low-pass filtering at 100 Hz on the EEG signal, removing outliers, and performing data supplementation by using linear interpolation;
(2.2) removing other bioelectrical components by using an independent component analysis (ICA) (a blind source separation algorithm), and then performing segmentation, baseline correction and common average reference; and
(2.3) performing filtering on the processed EEG signal at 0.3-3 Hz (δ wave), 4-8 Hz (θ wave), 8-13 Hz (α wave), 13-30 Hz (β wave) and 30-45 Hz (γ wave) by using a 4th-order Butterworth IIR filter, and sampling the EEG signal of each frequency by using the sliding time window with a length of 1 s and a step length of 100 ms to obtain a signal value of the corresponding time point.
(3) processing data of the each time point, and constructing a time-domain multi-layer brain network by taking an electrode as a node, a directed transfer function (DTF) as an intra-layer connection, and parameters of a weighted quadratic autoregressive (wSAR) model as an inter-layer connection. Particularly, a method for calculating a time-domain inter-layer connection is as follows: constructing the wSAR model: adding a weighted term to an autoregressive (AR) model based on a hypothesis that the closer a time distance is, the greater an impact becomes, and processing a constant term, and calculating parameters of the improved wSAR model as the inter-layer connection.

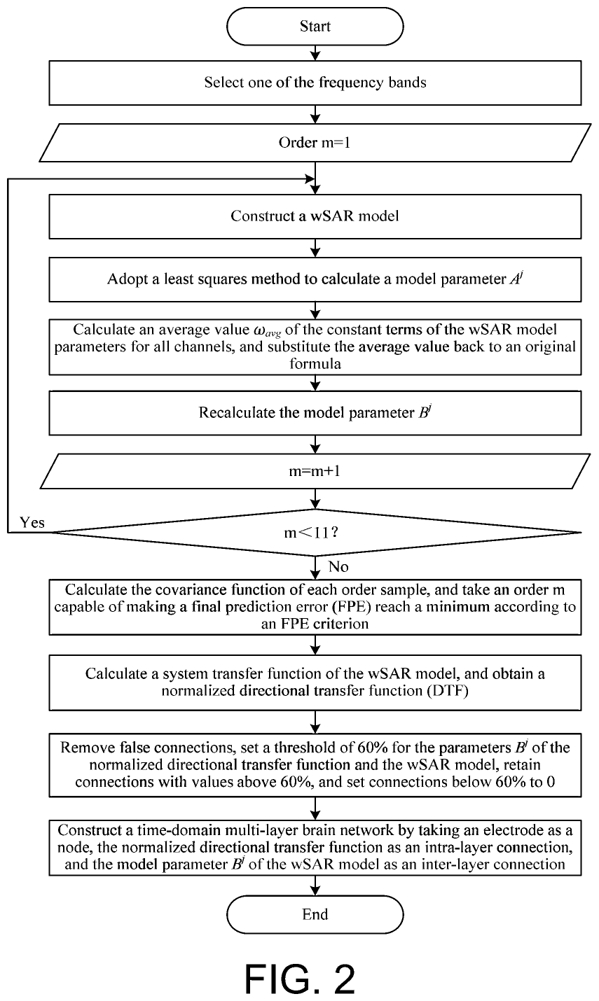

Specifically, as shown in FIG. 2, the step (3) includes following sub-steps:
(3.1) selecting an EEG signal of a certain channel in a certain frequency band, and constructing the wSAR model for the EEG signal:

$${x}_{i + 1}^{j} = {a}_{m}^{j} {x}_{i}^{j} + {a}_{m - 1}^{j} {x}_{i - 1}^{j} + … + {a}_{1}^{j} {x}_{i - m}^{j} + {ka}_{m}^{j} + {ω}^{j} + ε$$

where xᵢ^{j} represents an EEG signal value of a channel j at a moment i, aₘ^{j} represents a model parameter value of the channel j, ω^{j} represents a constant term, which is assumed to be an initial value of influence of each channel, ε represents a noise, a model order is m, and k is a weighted value. When a value of the k is too large, subsequent time terms will lose their meanings, therefore, in order to avoid the situation, k is set to an average value from xᵢ₋ₘ^{j} to xᵢ^{j}, and in order to consider an optimal order, an initial value of the model order m is set to 1 here.
(3.2) Rewriting a formula of the wSAR model as follows:

$$ε = {x}_{i + 1}^{j} - [ {a}_{m}^{j} ( {x}_{i}^{j} + k ) + {a}_{m - 1}^{j} {x}_{i - 1}^{j} + … + {a}_{1}^{j} {x}_{i - m}^{j} + {ka}_{m}^{j} + {ω}^{j} ]$$

a least squares method is adopted, and a partial derivative of ε² to a^{j} is set to equal to 0 to minimize a residual sum of squares.

In order to make the formula expression easier, the wSAR model is denoted in a matrix form as

$${Y}^{j} = [ \begin{matrix}{x}_{i + 1}^{j} \\ {x}_{i + 2}^{j} \\ … \\ {x}_{N}^{j}\end{matrix} ] , {X}^{j} = [ \begin{matrix}{x}_{i}^{j} + k & {x}_{i - 1}^{j} & … & {x}_{i - m}^{j} & 1 \\ {x}_{i + 1}^{j} + k & {x}_{i}^{j} & … & {x}_{i + 1 - m}^{j} & 1 \\ … & … & … & … & 1 \\ {x}_{N - 1}^{j} + k & {x}_{N - 2}^{j} & … & {x}_{N - 1 + m}^{j} & 1\end{matrix} ] , \\ {A}^{j} = [ \begin{matrix}{a}_{m}^{j} \\ … \\ {a}_{2}^{j} \\ {a}_{1}^{j} \\ {ω}^{j}\end{matrix} ] , ε = [ \begin{matrix}{ε}_{1} \\ {ε}_{2} \\ … \\ {ε}_{m}\end{matrix} ]$$

where N is a total number of time points, and a least squares estimation of a parameter matrix A^{j} is expressed as:

$${A}^{j} = {( {X}^{{j}^{T}} {X}^{j} )}^{- 1} {X}^{{j}^{T}} {Y}^{j}$$

(3.3) Obtaining all the parameter matrixes A^{j} by having EEG signals of all the channels in the frequency band calculated in the step (3.2). According to the actual situation, when neither action intention nor action execution is made, all the channels shall have a same preliminary value for an action response, therefore, preliminary values of all the channels need to be aligned. In the method, an average value of the constant term ω^{j} in each of the parameter matrix A^{j} is taken to obtain ω_{avg}, that is

$${ω}_{avg} = \frac{1}{M} {\sum }_{j} {ω}^{j}$$

where M represents a total number of the channels, and ω^{j} is a constant term of a parameter matrix of each of the channels. The step (3.2) is performed again by substituting ω_{avg} back to an original formula of the AR model of all the channels to obtain a final parameter matrix B^{j}.
(3.4) Taking the model order m from 1 to 10, repeating the steps (3.2) and (3.3), and calculating self-covariance functions σ₁, σ₂, . . . , σ₁₀ of samples of each order; and calculating an order capable of making a final prediction error (FPE) reach a minimum according to an FPE criterion, and selecting a parameter matrix B^{j} of the order.
(3.5) Multiplying both sides of an equation of the AR model of all the channels by a time-lag term xᵢ₋ₛ^{j}^{^{T}}, performing z transformation to obtain a system transfer function Hᵢⱼ(ƒ), which represents a transfer relationship from a channel i to the channel j; and then performing normalization to obtain a final directed transfer function (DTF) γᵢⱼ²(ƒ):

$${γ}_{i j}^{2} ( f ) = \frac{{\vert {H}_{ij} ( f ) \vert}^{2}}{{\sum }_{m = 1}^{M} {\vert {H}_{im} ( f ) \vert}^{2}}$$

where Hᵢₘ(ƒ) represents a transfer function from the channel i to a channel m.
(3.6) Performing calculation of the signals of all the frequency bands according to the steps (3.2)-(3.5) to finally construct the time-domain multi-layer brain network: taking electrodes as nodes; taking 60% of a threshold to remove false connections, and regarding the finally obtained γᵢⱼ²(ƒ) as the intra-layer connection, that is, a connection from a node i to a node j in a same layer is taken as γᵢⱼ²(ƒ); 60% of the threshold is taken to remove false connections, and the parameter matrix B^{j} is regarded as the intra-layer connection, that is, a connection between a node i in a k^{th} layer to a node i in an l^{th} layer of a frequency band j is b_{m+1-|k-l|}^{j}.
(4) Processing data of each frequency band, and calculating cross-frequency inter-layer coupling. A method for calculating a frequency-domain inter-layer connection is as follows: performing Hilbert transform on the EEG signal of each frequency, extracting amplitude and phase information, and calculating cross-frequency coupling (CFC) between every two frequencies, and selecting an average value of phase-amplitude coupling (PAC), phase-phase coupling (PPC), and amplitude-amplitude coupling (AAC) as a final inter-layer connection herein.

Specifically, the step (4) includes following sub-steps:
(4.1) selecting the EEG signal of each frequency, and performing the Hilbert transform on all the time points to obtain an amplitude value and a phase value of the each time point;
(4.2) calculating the PAC, the PPC, and the AAC between every two frequencies:

$$PAC = \vert \frac{1}{n} {\sum }_{i = 1}^{n} {a}_{i}^{H} {e}^{j {∅}_{i}^{L}} \vert$$

where n is a total number of time points in EEG data, aᵢ^{H} represents an amplitude value of a high-frequency band H at the moment i, and Øᵢ^{L} represents the phase of a low frequency band L at the moment i;

$$PPC = \vert \frac{1}{n} {\sum }_{i = 1}^{n} sgn [ Im ( {e}^{j ( {∅}_{i}^{a} - {∅}_{i}^{b} )} ) ] \vert$$

where Øᵢ^{a} and Øᵢ^{b} represent phase values of frequency bands a and b at the moment i, respectively, and an imaginary part and an sgn function are taken to reduce volume conduction effect and the influence of a common source;

AAC=Corr(Aⁱ,A^{j})
where Corr( ) represents a Pearson correlation coefficient, Aⁱ and A^{j} represent the amplitude values of frequency bands i and j, respectively; and
(4.3) calculating the average value of the PAC, the PPC and the AAC as a cross-frequency coupling (CFC) value, taking 60% of the threshold to remove the false connections, and using the CFC value as the inter-layer coupling between corresponding frequencies to form a frequency-domain part of the multi-layer brain network.
(5) Combining the obtained time-domain and frequency-domain multi-layer brain network respectively constructed in the steps (3) and (4) into a 3D standardized form: M=(V, E, ω, l), wherein V represent a set of points, E represents an edge set, ω represents a size of an edge, and l represents a position label of a point. In the standardized form, brain regions are abstracted into a z-axis, the time is an x-axis, and the frequency is a y-axis; that is, a single layer network is compressed into one dimension, and as a dimension of the number of layers increase, a number of dimensionalities of the multi-layer brain network increase.

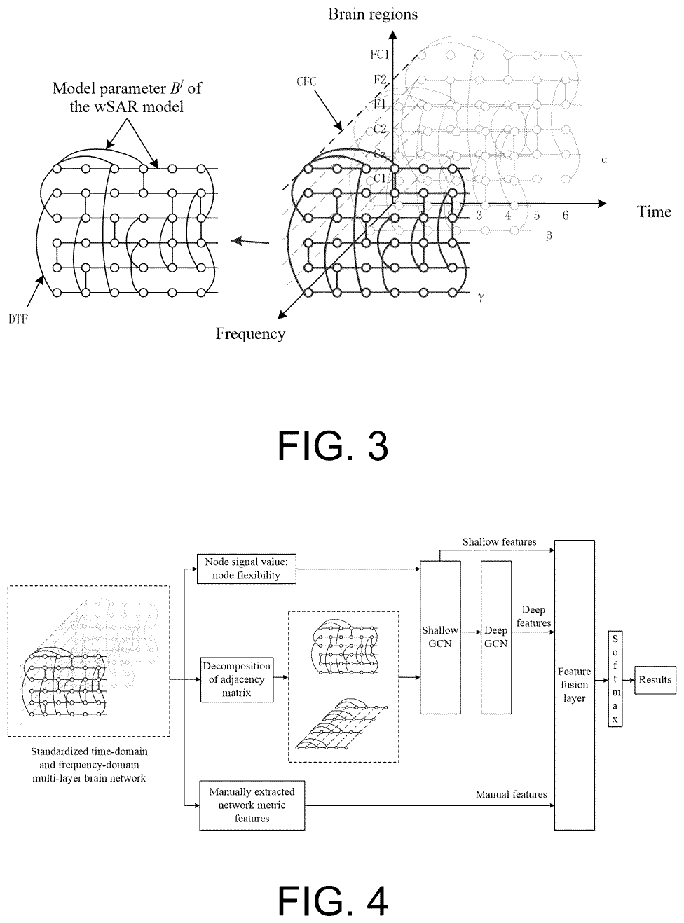

Specifically, as shown in FIG. 3, the step (5) includes following sub-steps:
The obtained time-domain and frequency-domain multi-layer brain network is written into the standardized form: M=(V, E, ω, l), where the brain regions are the z-axis, the time is the x-axis, and the frequency is the y-axis, as shown in FIG. 3, V is a point set, including a point label, for example, (“ν₁”, . . . , “ν_{N}”); E represents the edge, for example, (“e₁₂”, . . . , “e_{N(N-1)}”); ω is a connection value of the edge, for example (ω(νᵢ, νⱼ), . . . ); i is a position label of the point ν, which is used to indicate the position of the point, therefore, l is a 2D matrix, for example, ((“ν₁”, 1, 1, 1), . . . , (“ν₂₅”, 2, 5, 6), . . . ), and since the present disclosure has three dimensions, three numbers after the point label constitute coordinates of the point. A reason for standardizing the multi-layer brain network is to make it visualized and facilitate retrieval, and make it convenient for subsequent calculation of metrics and unified processing of a graph convolutional network (GCN).
(6) Calculating metrics of the multi-layer brain network in the standardized form obtained in the step (5), including a common multi-layer degree, eigenvector centrality, and temporal flexibility specific to the multi-layer brain network, which are used for a nerve analysis of natural action decoding, and finally, calculating an adjacency matrix, and inputting the standardized form into the GCN for classification and identification.

Specifically, as shown in FIG. 4, the step (6) includes following sub-steps:
(6.1) calculating metrics of the time-domain and frequency-domain multi-layer brain network: the multi-layer degree d(i), the eigenvector centrality θ_{jβ}, and a node flexibility F are used for a neurophysiological analysis. Furthermore, manually extracted features are inputted into a last layer of the GCN for weighted fusion of features:

$${d}_{M} ( i ) = {{\sum }_{j , k ∈ V} [}_{j , k \neq i} a ( i , j , k ) + a ( j , i , k ) + a ( k , i , j ) ]$$

$${ϑ}_{j β} = {λ}^{− 1} {M}_{j β}^{i a} {ϑ}_{j α}$$

$${F}_{j} = \frac{1}{N} {\sum }_{i} {f}_{i}$$

$$Q = {\sum }_{ijkr} [ ( {A}_{ijs} − {γ}_{s} {P}_{ijs} ) δ ( s , k ) + {ω}_{i s k} ] δ ( {c}_{ir} , {c}_{is} )$$

where d_{M}(i) is a total node degree of νᵢ, a(i,j,k) is an element in an adjacency tensor of the time-domain and frequency-domain multi-layer brain network; M_{jβ}^{iα} is the adjacency tensor of the time-domain and frequency-domain multi-layer brain network; θ_{jβ} and θ_{jα} are eigenvectors of the tensor corresponding to an eigenvalue λ; ƒᵢ is a number of changes of a node community at the each time point, N is a total number of changes, and Fⱼ is node flexibility of a node νⱼ; Q is a modular function used for community detection, where Aᵢⱼₛ is an adjacency matrix of an s^{th} layer, Pᵢⱼₛ is a relevant zero matrix, γₛ is a resolution parameter, ωᵢₛₖ is a connection value from a node i in the s^{th} layer to a node i in the k^{th} layer, cᵢᵣ and cᵢₛ are set parameters; and δ( ) is a Dirac function;
(6.2) inputting the time-domain and frequency-domain multi-layer brain network into the GCN, where the inputted node signal value is a degree of freedom of each node; and the inputted adjacency matrix is a super-adjacency matrix of a decomposed time-domain and frequency-domain multi-layer brain network. Particularly, a method for processing the super-adjacency matrix of a decomposed time-domain and frequency-domain multi-layer brain network is as follows: since the adjacency matrix of the time-domain and frequency-domain multi-layer brain network is a 3D tensor, and is difficult to be calculated due to a large number of dimensionalities. In order to reduce the computational load, inspired by Cartesian product of graphs, the time-domain and frequency-domain multi-layer brain network is decomposed into sub-networks in two directions, an original 3D adjacency matrix is decomposed into two orthogonal 2D super-adjacency matrices as adjacency matrix inputs to the GCN, and finally feature fusion is performed;
(6.3) a structure of the GCN includes two layers of GCN and one feature classification layer. In order to make full use of information of the time-domain and frequency-domain multi-layer brain network, the two layers of GCN are divided into a shallow layer of GCN and a deep layer of GCN, all graphs output features once after passing through the shallow layer of GCN, output features once again after passing through the deep layer of GCN, and finally, manual, shallow and deep features are fused to obtain final classification results.

It should be noted that the above content merely illustrates the technical idea of the present disclosure and cannot limit the protection scope of the present disclosure, those of ordinary skill in the art may also make some modifications and improvements without departing from the principle of the present disclosure, and these modifications and improvements should also fall within the protection scope of the claims of the present disclosure.

What is claimed is:

1. An EEG recognition method for a natural hand movement based on a time-domain and frequency-domain multi-layer brain network, comprising following steps:
(1) acquiring a multi-channel EEG signal of the natural hand movement;
(2) preprocessing the multi-channel EEG signal, extracting an EEG signal at each time point by using a sliding time window, and extracting a δ wave, a θ wave, a α wave, a β wave, and a γ wave at the each time point; and
(3) processing data of the each time point, and constructing a time-domain multi-layer brain network by taking an electrode as a node, a directed transfer function (DTF) as an intra-layer connection, and parameters of a weighted quadratic autoregressive (wSAR) model as an inter-layer connection; wherein a method for calculating a time-domain inter-layer connection is as follows: constructing the wSAR model: adding a weighted term to an autoregressive (AR) model based on a hypothesis that the closer a time distance is, the greater an impact becomes, and processing a constant term, and calculating parameters of the improved wSAR model as the inter-layer connection;
(4) processing data of each frequency band, and calculating cross-frequency inter-layer coupling; and a method for calculating a frequency-domain inter-layer connection is as follows: performing Hilbert transform on the EEG signal of each frequency, extracting amplitude and phase information, and calculating cross-frequency coupling (CFC) between every two frequencies, and selecting an average value of phase-amplitude coupling (PAC), phase-phase coupling (PPC), and amplitude-amplitude coupling (AAC) as a final inter-layer connection;
(5) combining the time-domain and frequency-domain multi-layer brain networks respectively constructed in the steps (3) and (4) into a 3D standardized form: M=(V, E, ω, l), wherein V represents a set of points, E represents an edge set, ω represents a size of an edge, and l represents a position label of a point; and in the standardized form, brain regions are abstracted into a z-axis, the time is an x-axis, the frequency is a y-axis, that is, a single-layer network is compressed into one dimension, and as a dimension of the number of layers increases, a number of dimensionalities of the multi-layer brain network increases; and
(6) calculating metrics of the multi-layer brain network in the standardized form obtained in the step (5), comprising a common multi-layer degree, eigenvector centrality, and temporal flexibility specific to the multi-layer brain network, which are used for a nerve analysis of natural action decoding; and finally, calculating an adjacency matrix, and inputting the standardized form into a graph convolutional network (GCN) for classification and identification.

2. The EEG recognition method for a natural hand movement based on a time-domain and frequency-domain multi-layer brain network according to claim 1, wherein the step (2) further comprises:
(2.1) performing low-pass filtering at 100 Hz on the EEG signal, removing outliers, and performing data supplementation by using linear interpolation;
(2.2) removing other bioelectrical components by using an independent component analysis (ICA) (a blind source separation algorithm), and then performing segmentation, baseline correction and common average reference; and
(2.3) performing filtering on the processed EEG signal at 0.3-3 Hz, 4-8 Hz, 8-13 Hz, 13-30 Hz and 30-45 Hz by using a 4th-order Butterworth IIR filter, and sampling the EEG signal of each frequency by using the sliding time window with a length of Is and a step length of 100 ms to obtain a signal value of the corresponding time point.

3. The EEG recognition method for a natural hand movement based on a time-domain and frequency-domain multi-layer brain network according to claim 1, wherein the step (3) further comprises:
(3.1) selecting an EEG signal of a certain channel in a certain frequency band, and constructing the wSAR model for the EEG signal:
$${x}_{i + 1}^{j} = {a}_{m}^{j} {x}_{i}^{j} + {a}_{m - 1}^{1} {x}_{i - 1}^{j} + … + {a}_{1}^{j} {x}_{i - m}^{j} + {ka}_{m}^{j} + {ω}^{j} + ε$$
in the formula, xᵢ^{j} represents an EEG signal value of a channel j at a moment i, aₘ^{j} represents a model parameter value of the channel j, ω^{j} represents a constant term, which is assumed to be an initial value of influence of each channel, ε represents a noise, a model order is m, and k is a weighted value; when a value of the k is too large, subsequent time terms will lose their meanings, therefore, in order to avoid the situation, k is set to an average value from xᵢ₋ₘ^{j} to xᵢ^{j}; and in order to consider an optimal order, an initial value of the model order m is set to 1 herein;
(3.2) rewriting a formula of the wSAR model as follows:
$$ε = {x}_{i + 1}^{j} - [ {a}_{m}^{j} ( {x}_{i}^{j} + k ) + {a}_{m - 1}^{j} {x}_{i - 1}^{j} + … + {a}_{i}^{j} {x}_{i - m}^{j} + {ka}_{m - 1}^{j} + {ω}^{j} ]$$
a least squares method is adopted, and a partial derivative of ε² to a^{j} is set to equal to 0 to minimize a residual sum of squares;
in order to make the formula expression easier, the wSAR model is denoted in a matrix form as
$${Y}^{j} = [ \begin{matrix}{x}_{i + 1}^{j} \\ {x}_{i + 2}^{j} \\ ⋯ \\ {x}_{N}^{j}\end{matrix} ] , \text{} {X}^{j} = [ \begin{matrix}{x}_{i}^{j} + k & {x}_{i − 1}^{j} & … & {x}_{i - m}^{j} & 1 \\ {x}_{i + 1}^{j} + k & {x}_{i}^{j} & … & {x}_{i + 1 - m}^{j} & 1 \\ ⋯ & ⋯ & … & … & 1 \\ {x}_{N − 1}^{j} + k & {x}_{N − 2}^{j} & ⋯ & {x}_{N − 1 + m}^{j} & 1\end{matrix} ] , \text{} {A}^{j} = [ \begin{matrix}{a}_{m}^{j} \\ ⋯ \\ {a}_{2}^{j} \\ {a}_{1}^{j} \\ {ω}^{j}\end{matrix} ] , ε = [ \begin{matrix}{ε}_{1} \\ {ε}_{2} \\ ⋯ \\ {ε}_{m}\end{matrix} ]$$
in the formula, Nis a total number of time points, and a least squares estimation of a parameter matrix A^{j} is expressed as:
$${A}^{j} = {( {X}^{{j}^{T}} {X}^{j} )}^{- 1} {X}^{{j}^{T}} {Y}^{j}$$
(3.3) obtaining all the parameter matrixes A^{j} by having EEG signals of all the channels in the frequency band calculated in the step (3.2); and taking an average value of the constant term ω^{j} in each of the parameter matrixes A^{j} to obtain ω_{avg}, that is
$${ω}_{aνg} = \frac{1}{M} {\sum }_{j} {ω}^{j}$$
in the formula, M represents a total number of the channels, and ω^{j} is a constant term of a parameter matrix of each of the channels; and substituting ω_{avg} back to an original formula of the AR model of all the channels, and performing the step (3.2) again to obtain a final parameter matrix B^{j};
(3.4) taking the model order m from 1 to 10, repeating the steps (3.2) and (3.3), and calculating self-covariance functions σ₁, σ₂, . . . , σ₁₀ of samples of each order; and calculating an order capable of making a final prediction error (FPE) reach a minimum according to an FPE criterion, and selecting a parameter matrix Bi of the order;
(3.5) multiplying both sides of an equation of the AR model of all the channels by a time-lag term xᵢ₋ₛ^{j}^{^{T}}, performing z transformation to obtain a system transfer function Hᵢⱼ(ƒ), which represents a transfer relationship from a channel i to the channel j; and then performing normalization to obtain a final directed transfer function (DTF) γᵢⱼ² (ƒ);
$${γ}_{ij}^{2} ( f ) = \frac{{\vert {H}_{ij} ( f ) \vert}^{2}}{{\sum }_{m = 1}^{M} {\vert {H}_{i m} ( f ) \vert}^{2}}$$
in the equation, Hᵢₘ(ƒ) represents a transfer function from the channel i to a channel m; and
(3.6) performing calculation of the signals of all the frequency bands according to the steps (3.2)-(3.5) to finally construct the time-domain multi-layer brain network: taking electrodes as nodes; taking 60% of a threshold to remove false connections, and regarding the finally obtained γᵢⱼ²(ƒ) as the intra-layer connection, that is, a connection from a node i to a node j in a same layer is taken as γᵢⱼ²(ƒ); 60% of the threshold is taken to remove false connections, and the parameter matrix B^{j} is regarded as the intra-layer connection, that is, a connection between a node i in a k^{th} layer to a node i in an l^{th} layer of a frequency band j is b_{m+1-|k-l|}^{j}.

4. The EEG recognition method for a natural hand movement based on a time-domain and frequency-domain multi-layer brain network according to claim 1, wherein the step (4) further comprises:
(4.1) selecting the EEG signal of each frequency, and performing the Hilbert transform on all the time points to obtain an amplitude value and a phase value of the each time point;
(4.2) calculating the PAC, the PPC, and the AAC between every two frequencies:
$$PAC = \vert \frac{1}{n} {\sum }_{i = 1}^{n} {a}_{i}^{H} {e}^{j {∅}_{i}^{L}} \vert$$
in the formula, n is a total number of time points in EEG data, aᵢ^{H} represents an amplitude value of a high-frequency band H at the moment i, and Øᵢ^{L} represents the phase of a low frequency band L at the moment i;
$$P PC = \vert \frac{1}{n} {\sum }_{i = 1}^{n} sgn [ Im ( {e}^{j ( {∅}_{i}^{a} {∅}_{i}^{b} )} ) ] \vert$$
in the formula, Øᵢ^{a} and [symbol] ᵢ^{b} represent phase values of frequency bands a and b at the moment i, respectively, and an imaginary part and an sgn function are taken to reduce volume conduction effect and the influence of a common source;
$$AAC = Corr ( {A}^{i} , {A}^{j} )$$
in the formula, Corr( ) represents a Pearson correlation coefficient, Aⁱ and A^{j} represent the amplitude values of frequency bands i and j, respectively; and
(4.3) calculating the average value of the PAC, the PPC and the AAC as a cross-frequency coupling (CFC) value, taking 60% of the threshold to remove the false connections, and using the CFC value as the inter-layer coupling between corresponding frequencies to form a frequency-domain part of the multi-layer brain network.

5. The EEG recognition method for a natural hand movement based on a time-domain and frequency-domain multi-layer brain network according to claim 1, wherein the step (5) further comprises:
the obtained time-domain and frequency-domain multi-layer brain network is written into the standardized form: M=(V, E, ω, l), wherein the brain regions are the z-axis, the time is the x-axis, and the frequency is the y-axis; and V is the set of points, comprising a point label; E represents the edge; ω is a connection value of the edge; i is a position label of the point ν, which is used to indicate the position of the point, therefore, l is a 2D matrix, and since the present disclosure has three dimensions, three numbers after the point label constitute coordinates of the point.

6. The EEG recognition method for a natural hand movement based on a time-domain and frequency-domain multi-layer brain network according to claim 1, wherein the step (6) further comprises:
(6.1) calculating metrics of the time-domain and frequency-domain multi-layer brain network: the multi-layer degree d(i), the eigenvector centrality θ_{jβ}, and a node flexibility F are used for a neurophysiological analysis; and furthermore, manually extracted features are inputted into a last layer of the GCN for weighted fusion of features:
$${d}_{M} ( i ) = {{\sum }_{j , k ∈ V} [}_{j , k \neq i} a ( i , j , k ) + a ( j , i , k ) + a ( k , i , j ) ]$$
$${ϑ}_{j β} = {λ}^{− 1} {M}_{j β}^{i a} {ϑ}_{j α}$$
$${F}_{j} = \frac{1}{N} {\sum }_{i} {f}_{i}$$
$$Q = {\sum }_{ijkr} [ ( {A}_{ijs} − {γ}_{s} {P}_{ijs} ) δ ( s , k ) + {ω}_{i s k} ] δ ( {c}_{ir} , {c}_{is} )$$
in the formula, d_{M}(i) is a total node degree of νᵢ, a(i,j,k) is an element in an adjacency tensor of the time-domain and frequency-domain multi-layer brain network; M_{jβ}^{iα} is the adjacency tensor of the time-domain and frequency-domain multi-layer brain network; θ_{jβ} and θ_{jα} are eigenvectors of the tensor corresponding to an eigenvalue Δ; ƒᵢ is a number of changes of a node community at the each time point, N is a total number of changes, and Fⱼ is node flexibility of a node νⱼ; Q is a modular function used for community detection, wherein Aᵢⱼₛ is an adjacency matrix of an s^{th} layer, Pᵢⱼₛ is a relevant zero matrix, γₛ is a resolution parameter, ωᵢₛₖ is a connection value from a node i in the s^{th} layer to a node i in the k^{th} layer, cᵢᵣ and cᵢₛ are set parameters; and δ( ) is a Dirac function;
(6.2) inputting the time-domain and frequency-domain multi-layer brain network into the GCN, wherein the inputted node signal value is a degree of freedom of each node; and the inputted adjacency matrix is a super-adjacency matrix of a decomposed time-domain and frequency-domain multi-layer brain network; particularly, a method for processing the super-adjacency matrix of a decomposed time-domain and frequency-domain multi-layer brain network is as follows: since the adjacency matrix of the time-domain and frequency-domain multi-layer brain network is a 3D tensor, and is difficult to be calculated due to a large number of dimensionalities;
and in order to reduce the computational load, inspired by Cartesian product of graphs, the time-domain and frequency-domain multi-layer brain network is decomposed into sub-networks in two directions, an original 3D adjacency matrix is decomposed into two orthogonal 2D super-adjacency matrices as adjacency matrix inputs to the GCN, and finally feature fusion is performed; and
(6.3) a structure of the GCN comprises two layers of GCN and one feature classification layer; and in order to make full use of information of the time-domain and frequency-domain multi-layer brain network, the two layers of GCN are divided into a shallow layer of GCN and a deep layer of GCN, all graphs output features once after passing through the shallow layer of GCN, output features once again after passing through the deep layer of GCN, and finally, manual, shallow and deep features are fused to obtain final classification results.
